(19) United States
(12) Patent Application Publication
(10) Pub. No.: US 2007/0009882 A1
(43) Pub. Date: Jan. 11, 2007

(54) In vitro platform for screening agents inducing islet cell neogenesis

(75) Inventor: Lawrence Rosenberg, Montreal (CA)

Correspondence Address: BERESKIN AND PARR, 40 KING STREET WEST, BOX 401, TORONTO, ON, M5H 3Y2, CA

(73) Assignee: McGill University, Montreal, QC (CA)

(21) Appl. No.: 10/558,191

(22) PCT Filed: May 27, 2004
(86) PCT No.: PCT/CA04/00788

§ 371 Date: Nov. 25, 2005

Related U.S. Application Data
(60) Provisional application No. 60/473,153, filed on May 27, 2003.

(51) Int. Cl. C12N 5/08; C12Q 1/00

(52) U.S. Cl. 435/4; 435/366

(57) ABSTRACT

The present invention relates to an in vitro method for screening agents inducing islet cell neogenesis or duct-to-islet cell transdifferentiation, which comprises the steps of: a) expanding in vitro cells of a duct-like structure obtained by inducing cystic formation in cells in or associated with post-natal islets of Langerhans; b) treating said expanded cells of said duct-like structure with an agent screened; and c) determining potency of said agent of inducing islet cell differentiation of said duct-like structure in becoming insulin-producing cells.

### BACKGROUND OF THE INVENTION

(a) Field of the Invention

The invention relates to an in vitro platform for identifying potential compounds as being capable of inducing islet cell neogenesis or duct-to-islet cell transdifferentiation.

(b) Description of Prior Art

Diabetes Mellitus

Diabetes mellitus has been classified as type I, or insulin-dependent diabetes mellitus (IDDM) and type II, or non-insulin-dependent diabetes mellitus (NIDDM). NIDDM patients have been subdivided further into (a) nonobese (possibly IDDM in evolution), (b) obese, and (c) maturity onset (in young patients). Among the population with diabetes mellitus, about 20% suffer from IDDM. Diabetes develops either when a diminished insulin output occurs or when a diminished sensitivity to insulin cannot be compensated for by an augmented capacity for insulin secretion. In patients with IDDM, a decrease in insulin secretion is the principal factor in the pathogenesis, whereas in patients with NIDDM, a decrease in insulin sensitivity is the primary factor. The mainstay of diabetes treatment, especially for type I disease, has been the administration of exogenous insulin.

Rationale for More Physiologic Therapies

Tight glucose control appears to be the key to the prevention of the secondary complications of diabetes. The results of the Diabetes Complications and Control Trial (DCCT), a multicenter randomized trial of 1441 patients with insulin dependent diabetes, indicated that the onset and progression of diabetic retinopathy, nephropathy, and neuropathy could be slowed by intensive insulin therapy (The Diabetes Control and Complication Trial Research Group, N. Engl. J. Med., 1993; 29:977-986). Strict glucose control, however, was associated with a three-fold increase in incidence of severe hypoglycemia, including episodes of seizure and coma. As well, although glycosylated hemoglobin levels decreased in the treatment group, only 5% maintained an average level below 6.05% despite the enormous amount of effort and resources allocated to the support of patients on the intensive regime (The Diabetes Control and Complication Trial Research Group, N. Engl. J. Med., 1993; 29:977-986). The results of the DCCT clearly indicated that intensive control of glucose can significantly reduce (but not completely protect against) the long-term microvascular complications of diabetes mellitus.

Other Therapeutic Options

The delivery of insulin in a physiologic manner has been an elusive goal since insulin was first purified by Banting, Best, McLeod and Collip. Even in a patient with tight glucose control, however, exogenous insulin has not been able to achieve the glucose metabolism of an endogenous insulin source that responds to moment-to-moment changes in glucose concentration and therefore protects against the development of microvascular complications over the long term.

A major goal of diabetes research, therefore, has been the development of new forms of treatment that endeavor to reproduce more closely the normal physiologic state. One such approach, a closed-loop insulin pump coupled to a glucose sensor, mimicking β-cell function in which the secretion of insulin is closely regulated, has not yet been successful. Only total endocrine replacement therapy in the form of a transplant has proven effective in the treatment of diabetes mellitus. Although transplants of insulin-producing tissue are a logical advance over subcutaneous insulin-injections, it is still far from clear whether the risks of the intervention and of the associated long-term immunosuppressive treatment are lower those in diabetic patients under conventional treatment.

Despite the early evidence of the potential benefits of vascularized pancreas transplantation, it remains a complex surgical intervention, requiring the long-term administration of chronic immunosuppression with its attendant side effects. Moreover, almost 50% of successfully transplanted patients exhibit impaired tolerance curves (Wright F H et al., Arch. Surg., 1989;124:796-799; Landgraft R et al., Diabetologia 1991; 34 (suppl 1):S61; Morel P et al., Transplantation 1991; 51:990-1000), raising questions about their protection against the long-term complications of chronic hyperglycemia.

The major complications of whole pancreas transplantation, as well as the requirement for long term immunosuppression, has limited its wider application and provided impetus for the development of islet transplantation. Theoretically, the transplantation of islets alone, while enabling tight glycemic control, has several potential advantages over whole pancreas transplantation. These include the following: (i) minimal surgical morbidity, with the infusion of islets directly into the liver via the portal vein; (ii) the possibility of simple retransplantation for graft failures; (iii) the exclusion of complications associated with the exocrine pancreas; (iv) the possibility that islets are less immunogenic, eliminating the need for immunosuppression and enabling early transplantation into non-uremic diabetics; (v). the possibility of modifying islets in vitro prior to transplantation to reduce their immunogenicity; (vi) the ability to encapsulate islets in artificial membranes to isolate them from the host immune system; and (vii) the related possibility of using xenotransplantation of islets immunoisolated as part of a biohybrid system. Moreover, they permit the banking of the endocrine cryopreserved tissue and a careful and standardized quality control program before the implantation.

The Problem of Islet Transplantation

Adequate numbers of isogenetic islets transplanted into a reliable implantation site can only reverse the metabolic abnormalities in diabetic recipients in the short term. In those that were normoglycemic post-transplant, hyperglycemia recurred within 3-12 mo. (Orloff M, et. al., Transplantation 1988; 45:307). The return of the diabetic state that occurs with time has been attributed either to the ectopic location of the islets, to a disruption of the enteroinsular axis, or to the transplantation of an inadequate islet cell mass (Bretzel R G, et al. In: Bretzel R G, (ed) Diabetes mellitus. (Berlin: Springer, 1990) p.22-9).

Studies of the long term natural history of the islet transplant, that examine parameters other than graft function, are few in number. Only one report was found in which an attempt was specifically made to study graft morphology (Alejandro R, et. al., J Clin Invest 1986; 78: 1339). In that study, purified islets were transplanted into the canine liver via the portal vein. During prolonged follow-up, delayed failures of graft function occurred. Unfortunately, the graft was only examined at the end of the study, and not over time as function declined. Delayed graft failures have also been confirmed by other investigators for dogs (Warnock G L et. al., Can. J. Surg., 1988; 31: 421 and primates (Sutton R, et. al., Transplant Proc., 1987; 19: 3525). Most failures are presumed to be the result of rejection despite appropriate immunosuppression.

Because of these failures, there is currently much enthusiasm for the immunoisolation of islets, which could eliminate the need for immunosuppression. The reasons are compelling. Immunosuppression is harmful to the recipient, and may impair islet function and possibly cell survival (Metrakos P, et al., J. Surg. Res., 1993; 54: 375). Unfortunately, micro-encapsulated islets injected into the peritoneal cavity of the dog fail within 6 months (Soon-Shiong P, et. al., Transplantation 1992; 54: 769), and islets placed into a vascularized biohybrid pancreas also fail, but at about one year. In each instance, however, histological evaluation of the graft has indicated a substantial loss of islet mass in these devices (Lanza R P, et. al., Diabetes 1992; 41: 1503). No reasons have been advanced for these changes. Therefore maintenance of an effective islet cell mass post-transplantation remains a significant problem.

In addition to this unresolved issue, is the ongoing problem of the lack of source tissue for transplantation. The number of human donors is insufficient to keep up with the potential number of recipients. Moreover, given the current state of the art of islet isolation, the number of islets that can be isolated from one pancreas is far from the number required to effectively reverse hyperglycemia in a human recipient.

In response, three competing technologies have been proposed and are under development. First, islet cryopreservation and islet banking. The techniques involved, though, are expensive and cumbersome, and do not easily lend themselves to widespread adoption. In addition, islet cell mass is also lost during the freeze-thaw cycle. Therefore this is a poor long-term solution to the problem of insufficient islet cell mass. Second, is the development of islet xenotransplantation. This idea has been coupled to islet encapsulation technology to produce a biohybrid implant that does not, at least in theory, require immunosuppression. There remain many problems to solve with this approach, not least of which, is that the problem of the maintenance of islet cell mass in the post-transplant still remains. Third, is the resort to human fetal tissue, which should have a great capacity to be expanded ex vivo and then transplanted. However, in addition to the problems of limited tissue availability, immunogenicity, there are complex ethical issues surrounding the use of such a tissue source that will not soon be resolved. However, there is an alternative that offers similar possibilities for near unlimited cell mass expansion.

An entirely novel approach, proposed by Rosenberg in 1995 (Rosenberg L et al., Cell Transplantation, 1995;4:371-384), was the development of technology to control and modulate islet cell neogenesis and new islet formation, both in vitro and in vivo. The concept assumed that (a) the induction of islet cell differentiation was in fact controllable; (b) implied the persistence of a stem cell-like cell in the adult pancreas; and (c) that the signal(s) that would drive the whole process could be identified and manipulated.

In a series of in vivo studies, Rosenberg and co-workers established that these concepts were valid in principle, in the in vivo setting (Rosenberg L et al., Diabetes, 1988;37:334-341; Rosenberg L et al., Diabetologia, 1996;39:256-262), and that diabetes could be reversed.

The well known teachings of in vitro islet cell expansion from a non-fetal tissue source comes from Peck and co-workers (Corneliu J G et al., Horm. Metab. Res., 1997;29:271-277), who describe isolation of a pluripotent stem cell from the adult mouse pancreas that can be directed toward an insulin-producing cell. These findings have not been widely accepted. First, the result has not proven to be reproducible. Second, the so-called pluripotential cells have never been adequately characterized with respect to phenotype. And third, the cells have certainly not been shown to be pluripotent.

More recently two other competing technologies have been proposed the use of engineered pancreatic β-cell lines (Efrat S, Advanced Drug Delivery Reviews, 1998;33:45-52), and the use of pluripotent embryonal stem cells (Shamblott M J et al., Proc. Natl. Acad. Sci. USA, 1998;95:13.726-13731). The former option, while attractive, is associated with significant problems. Not only must the engineered cell be able to produce insulin, but it must respond in a physiologic manner to the prevailing level of glucose- and the glucose sensing mechanism is far from being understood well enough to engineer it into a cell. Many proposed cell lines are also transformed lines, and therefore have a neoplastic potential. With respect to the latter option, having an embryonal stem cell in hand is appealing because of the theoretical possibility of being able to induce differentiation in any direction, including toward the pancreatic β-cell. However, the signals necessary to achieve this milestone remain unknown.

To date, the only in vitro test system for examining the bioactivity of compounds on duct cells that exists, is a duct cell proliferation assay based on hamster pancreatic cells. Cell proliferation is different than islet cell neogenesis.

The only way available to measure islet cell neogenesis is an in vivo method involving live animals (and not humans).

It would be highly desirable to be provided with an in vitro platform for identifying potential compounds as being capable of inducing islet cell neogenesis or duct-to-islet cell transdifferentiation. Agents identified according to the platform of the present invention could be used in diabetes therapies, such as for the preparation of dedifferentiated cells derived from post-natal islets of Langerhans, their expansion and the guided induction of islet cell differentiation, leading to insulin-producing cells that can be used for the treatment of diabetes mellitus. Furthermore, it would be desirable to be provided with such a platform using human cells.

### SUMMARY OF THE INVENTION

One aim of the invention is to provide an in vitro platform for identifying potential compounds as being capable of inducing islet cell neogenesis or duct-to-islet cell transdifferentiation.

In accordance with one embodiment of the present invention there is provided an in vitro platform for screening agents inducing islet cell neogenesis or duct-to-islet cell transdifferentiation, which comprises the steps of:

a) expanding in vitro cells of a duct-like structure obtained by inducing cystic formation in cells in or associated with post-natal islets of Langerhans; and

b) treating said expanded cells of said duct-like structure with an agent being screened; and

c) determining potency of said agent of inducing islet cell differentiation of said duct-like structure in becoming insulin-producing cells.

Preferably, step a) and step b) are concurrently effected using a solid matrix (such as 3-D collagen type-1 gel matrix), basal feeding medium (such as DMEM/F12 medium) and appropriate growth factors (such as EGF and cholera toxin) to permit the development, maintenance and expansion of a dedifferentiated cell population.

Preferably, the cells used are human cells.

In accordance with one embodiment of the present invention there is provided an islet cell culture, which comprises insulin-produding islet cells in a suitable culture medium, wherein said islet cells are characterized.

The islet cell culture may be characterized in a genetic, an immunologic or a genomic manner.

The characterization may be effected using a DNA microarray analysis.

In accordance with one embodiment of the present invention there is provided an in vitro method for evaluating biological effects of agents on islet cells, which comprises the steps of:

a) treating the islet cell culture of the present invention with an agent being evaluated for a time sufficient for a biological effect to be occurring; and

b) determining biological effect of the agent on islet cells by monitoring changes in insulin production compared to a standard curve obtained with a control islet cell culture.

The agent may selected from the group consisting of immunosuppressive agents, growth factors and anti-apoptotic agents.

For the purpose of the present invention the following terms are defined below.

The expression “post-natal islets of Langerhans” is intended to mean islet cells and associated cells, such as duct cells, of any origin, such as human, porcine and canine, among others.

The expression “dedifferentiated cells” is intended to mean cells of any origin which are stem/progenitor like cells.

### BRIEF DESCRIPTION OF THE DRAWINGS

FIG. 1 illustrates Islet-Duct transformation at isolation day, day 3 and day 10;

FIG. 2 illustrates Drug A-mediated Duct-to-Islet transformation;

FIG. 3 illustrates the % transformation of Drug A vs control in Duct-to-Islet Differenciation;

FIG. 4 illustrates the % of total cells for CK-19 control, CK-19 Drug A, PDX1 control and PDX1 DRUG A in CK-19/PDX1 immunoreactivity essay;

FIG. 5 illustrates the % of total area for insulin, glucagon and somatostatin in Control and Drug A in Islet hormone immunoreactivity essay;

FIG. 6 illustrates the insulin secretion in basal media, control and Drug A;

FIG. 7 illustrates cellular immunoreactivites of new islets derived from ducts;

FIG. 8A illustrates cellular proliferation in control and Drug A;

FIG. 8B illustrates cellular apoptosis in control and Drug A;

FIG. 9 illustrates the regulation of islet differentiation;

FIG. 10A illustrates Caspase-3 activity at Day 10, Day 14 control and Day 14 Drug A; and

FIG. 10B illustrates JNK activity at Day 10, Day 14 control and Day 14 Drug A.

### DETAILED DESCRIPTION OF THE INVENTION

Transdifferentiation is a change from one differentiated phenotype to another, involving morphological and functional phenotypic markers (Okada T S., Develop. Growth and Differ. 1986;28:213-321). The best-studied example of this process is the change of amphibian iridial pigment cells to lens fibers, which proceeds through a sequence of cellular dedifferentiation, proliferation and finally redifferentiation (Okada T S, Cell Diff. 1983;13:177-183; Okada T S, Kondoh H, Curr. Top Dev. Biol., 1986;20:1-433; Yamada T, Monogr. Dev. Biol., 1977;13:1-124). Direct transdifferentiation without cell division has also been reported, although it is much less common (Beresford Wash., Cell Differ. Dev., 1990;29:81-93). While transdifferentiation has been thought to be essentially irreversible, i.e. the transdifferentiated cell does not revert back into the cell type from which it arose, this has recently been reported not to be the case (Danto S I et al., Am. J. Respir. Cell Mol. Biol., 1995;12:497-502). Nonetheless, demonstration of transdifferentiation depends on defining in detail the phenotype of the original cells, and on proving that the new cell type is in fact descended from cells that were defined (Okada T S, Develop. Growth and Differ. 1986;28:213-321).

In many instances, transdifferentiation involves a sequence of steps. Early in the process, intermediate cells appear that express neither the phenotype of the original nor the subsequent differentiated cell types, and therefore they have been termed dedifferentiated. The whole process is accompanied by DNA replication and cell proliferation. Dedifferentiated cells are assumed a priori to be capable of forming either the original or a new cell type, and thus are multipotential (Itoh Y, Eguchi G, Cell Differ., 1986;18:173-182; Itoh Y, Eguchi G, Develop. Biology, 1986;115:353-362; Okada T S, Develop. Growth and Differ, 1986;28:213-321).

Stability of the cellular phenotype in adult organisms is probably related to the extracellular milieu, as well as cytoplasmic and nuclear components that interact to control gene expression. The conversion of cell phenotype is likely to be accomplished by selective enhancement of gene expression, which controls the terminal developmental commitment of cells.

The pancreas is composed of several types of endocrine and exocrine cells, each responding to a variety of trophic influences. The ability of these cells to undergo a change in phenotype has been extensively investigated because of the implications for the understanding of pancreatic diseases such as cancer and diabetes mellitus. Transdifferentiation of pancreatic cells was first noted nearly a decade ago. Hepatocyte-like cells, which are normally not present in the pancreas, were observed following the administration of carcinogen (Rao M S et al., Am. J. Pathol., 1983;110:89-94; Scarpelli D G, Rao M S, Proc. Nat. Acad. Sci. USA 1981;78:2577-2581) to hamsters and the feeding of copper-depleted diets to rats (Rao M S, et al., Cell Differ., 1986;18:109-117). Recently, transdifferentiation of isolated acinar cells into duct-like cells has been observed by several groups (Arias A E, Bendayan M, Lab Invest., 1993;69:518-530; Hall P A, Lemoine N R, J. Pathol., 1992;166:97-103; Tsao M S, Duguid W P, Exp. Cell Res., 1987;168:365-375). In view of these observations it is probably germane that during embryonic development, the hepatic and pan-creatic anlagen are derived from a common endodermal.

In accordance with one embodiment of the present invention, the platform technology is based on a combination of observations, incorporating the following components that are necessary and sufficient for the preparation of dedifferentiated intermediate cells from adult pancreatic islets of Langerhans:
1. a solid matrix permitting “three dimensional” culture;
2. the presence of matrix proteins including but not limited to collagen type I and laminin; and
3. the growth factor EGF and promoters of cAMP, including but not limited to cholera toxin and forskolin.

The preferred feeding medium is DMEM/F12 with 10% fetal calf serum. In addition, the starting tissue must be freshly isolated and cultured without absolute purification.

The use of a matrix protein-containing solid gel is an important part of the culture system, because extracellular matrix may promote the process of transdifferentiation. This point is highlighted by isolated pancreatic acinar cells, which transdifferentiate to duct-like structures when entrapped in Matrigel basement membrane (Arias A E, Bendayafn M, Lab Invest., 1993;69:518-530), or by retinal pigmented epithelial cells, which transdifferentiate into neurons when plated on laminin-containing substrates (Reh T A et al., Nature 1987;330:68-71). Most recently, Gittes et al. demonstrated, using 11-day embryonic mouse pancreas, that the default path for growth of embryonic pancreatic epithelium is to form islets (Gittes G K et al., Development 1996; 122:439-447). In the presence of basement membrane constituents, however, the pancreatic anlage epithelium appears to programmed to form ducts. This finding again emphasizes the interrelationship between ducts and islets and highlights the important role of the extracellular matrix.

This completes stage 1 (the production of dedifferentiated intermediate cells) of the process. During the initial 96 h of culture, islets undergo a cystic transformation associated with (Arias A E, Bendayan M, Lab. Invest., 1993;69:518-530) a progressive loss of insulin gene expression, (2) a loss of immunoreactivity for insulin protein, and (3) the appearance of CKA 19, a marker for ductal cells. After transformation is complete, the cells have the ultrastructural appearance of primitive duct-like cells. Cyst enlargement after the initial 96 h is associated, at least in part, with a tremendous increase in cell replication. These findings are consistent with the transdifferentiation of an islet cell to a ductal cell (Yuan et al., Differentiation, 1996; 61:67-75).

Evidence for the return to an islet cell phenotype includes: (1) the re-appearance of solid spherical structures; (2) loss of CK-19 expression; (3) the demonstration of endosecretory granules on electron microscopy; (4) the re-appearance of pro-insulin mRNA on in situ hybridization; (5) the return of a basal release of insulin into the culture medium.

The present invention will be more readily understood by referring to the following examples which are given to illustrate the invention rather than to limit its scope.

### EXAMPLE I

### Islet Isolation and Purification

Pancreata from six mongrel dogs of both sexes (body weight 25-30 kg) were resected under general anesthesia in accordance with Canadian Council for Animal Care guidelines (Wang R N, Rosenberg L (1999) J Endocrology 163 181-190). Prior to removal, the pancreatic ducts were cannulated to permit intraductal infusion with Liberase CI® (1.25 mg/ml) (Boehringer Mannheim, Indianapolis, Ind., USA) according to established protocols (Horaguchi A, Merrell R C (1981) Diabetes 30 455-461; Ricordi C (1992) Pancreatic islet cell transplantation. pp 99-112. Ed Ricordi C. Austin: R. G. Landes Co.). Purification was achieved by density gradient separation in a three-step EuroFicoll gradient using a COBE 2991 Cell Processor (COBE BCT, Denver, Colo., USA) (London NJM et al. (1992) Pancreatic islet cell transplantation. pp 113-123. Ed Ricordi C. Austin: R. G. Landes Co.). The final preparation consisted of 95% dithizone-positive structures with diameters ranging from 50 to 500 μm.

### EXAMPLE II

### Screening compounds for Islet Neogenesis potency

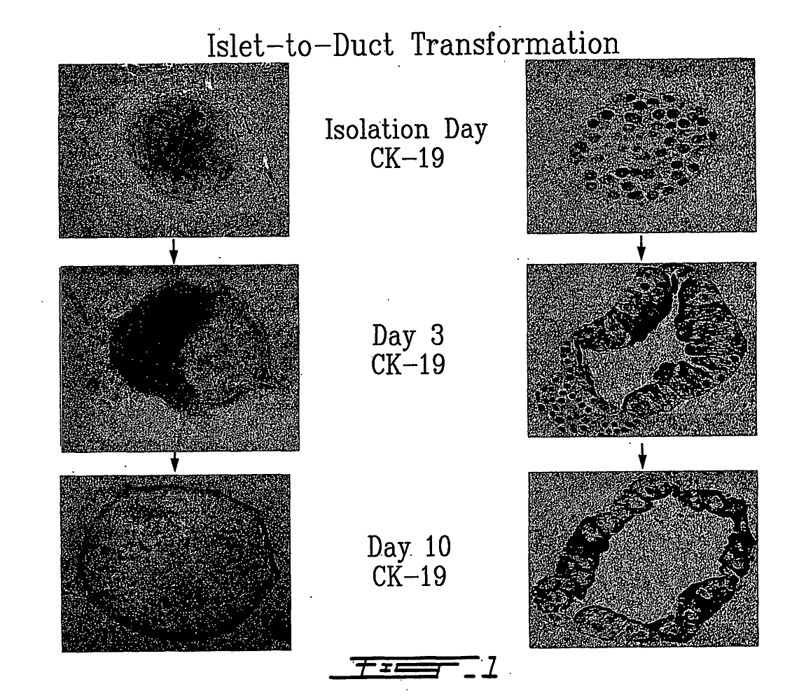

Adult islets were isolated, where each preparation used was over 95% pure, and transformed 100% of these islets into duct epithelial structures under defined culture conditions. The panel on the left of FIG. 1 is from an inverted microscope and follows a typical islet as it transforms over a 10-day period to a duct epithelial structure. During this transformation process, the appearance, as shown on the panel on the right of FIG. 1, of the duct epithelial cell marker CK-19 in every cell of the new ductal structures formed indicates a phenotypic switch from islet to duct. Also, there was a complete loss of islet cell hormone expression in all of these duct cells.

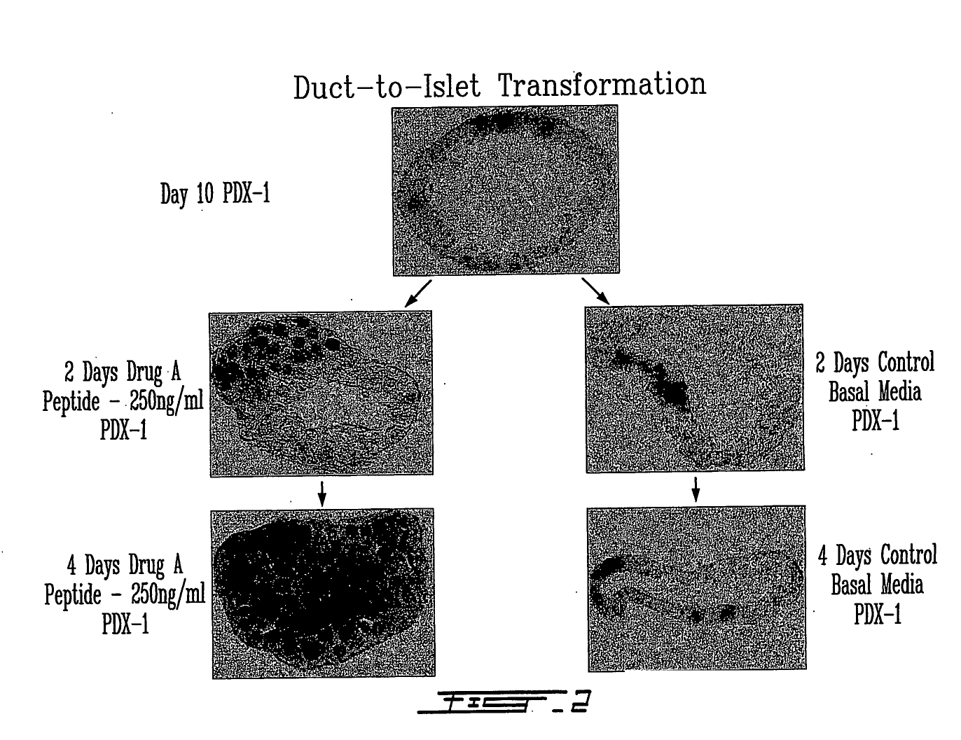

This islet-to-duct model was then used to study the effects of Drug A on this homogeneous population of duct epithelial cells. As you can see from the panels on the left of FIG. 2, after 4 days of Drug A treatment at a concentration 250 ng/ml, complete islet formation is from the duct was associated with a tremendous increase in PDX-1 expression (3-fold). PDX-1 is a transcription factor associated with islet development and differentiation during pancreatic development. In the control group seen on the right panel of FIG. 2, which was supplemented with only basal media for 4 days, none of the ducts transformed into islets and there was no increase in PDX-1 expression. What being obtained then here is an in vitro model which mirrors fetal ontogeny of the pancreas where there is new islet formation from the ductal epithelium—and it is shown here that this process is inducible in the adult pancreas using Drug A.

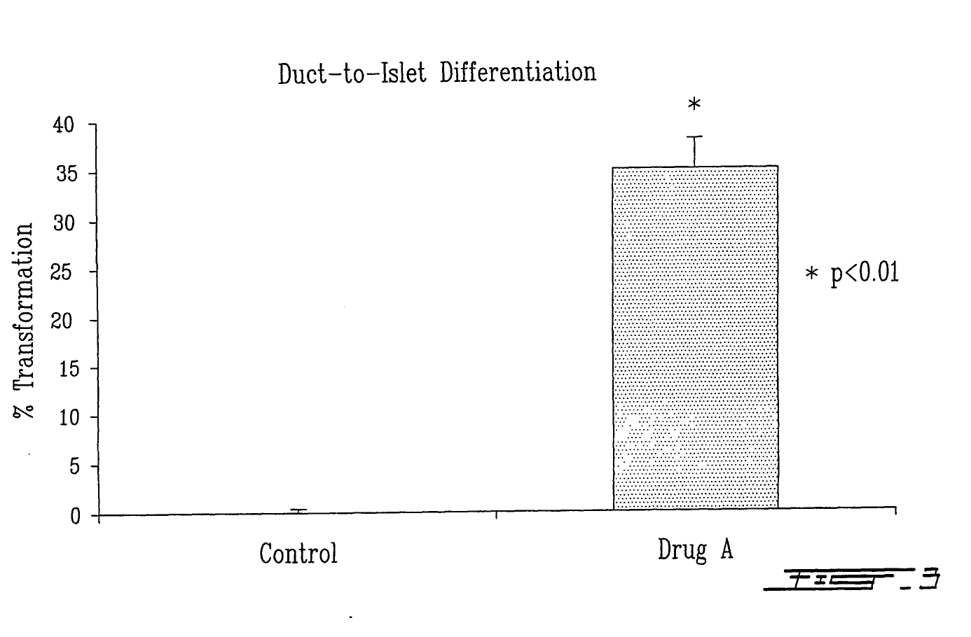

Of the total amount of ductal structures which accounted for 100% of the tissue in culture, 35% of these structures differentiated into islets after 4 days of Drug A treatment whereas none of the ducts in the control group differentiated into islets as illustrated in FIG. 3.

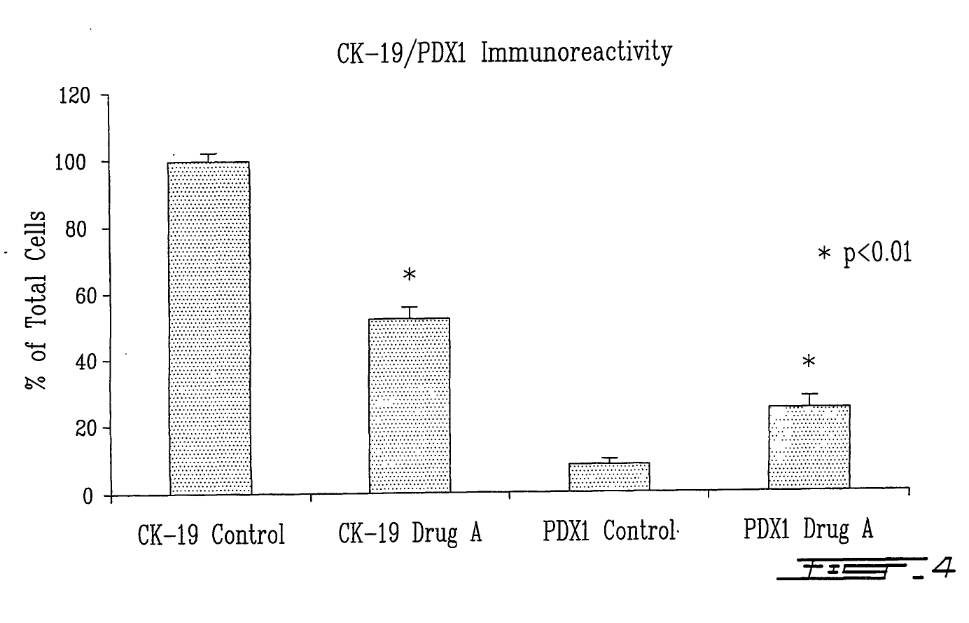

FIG. 4 illustrates that during duct-to-islet transformation, 100% of all cells in the control group continued to express the duct epithelial cell marker CK-19 whereas there was a loss of CK-19 expression after 4-day Drug A treatment as only ½ of these cells expressed CK-19. Also, 4-day Drug A treatment led to a 3-fold increase in PDX-1 expression in all treated cells whereas no increase was seen in the control Group.

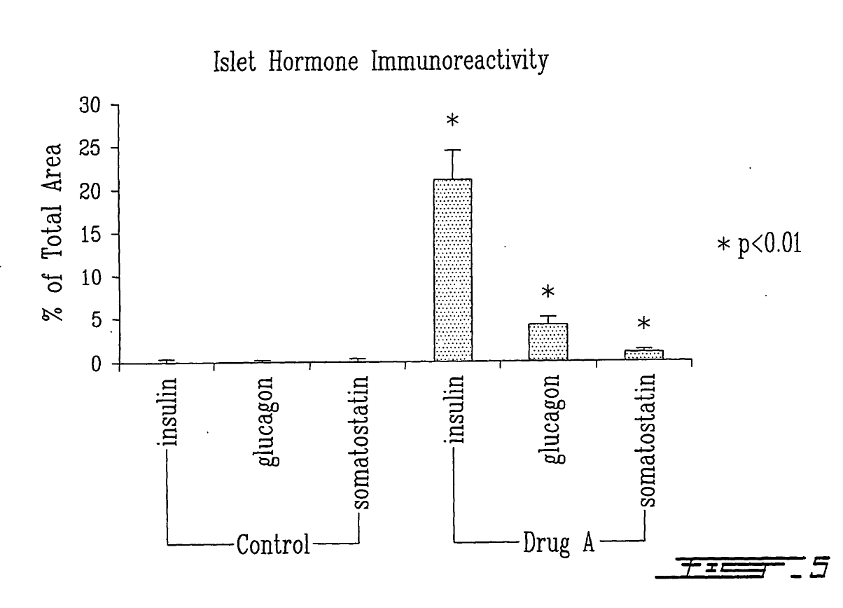

FIG. 5 illustrates that islet cell hormones, insulin, glucagon and somatostatin were all undetectable in all of the cells in the control group after 4 days whereas in the DRUG A group, the presence of these islet cell hormones was detected using immunohistochemistry and were found to be expressed in the same proportions as in normal adult human islets.

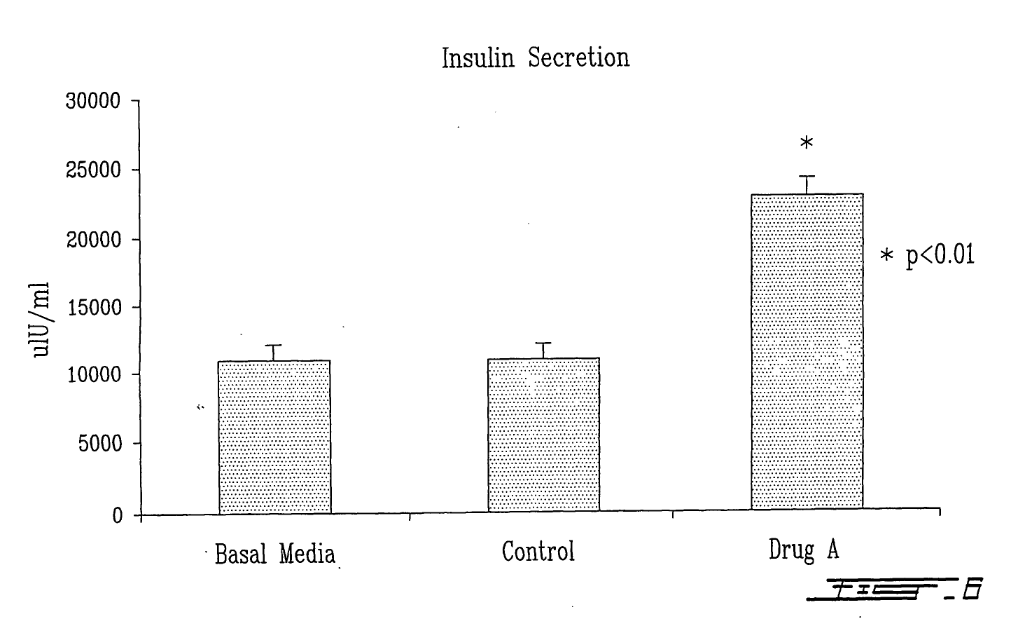

FIG. 6 illustrates that insulin secretion, as measured by ELISA, also increased significantly after Drug A treatment. The control group did not appear to secrete any insulin as the amount measured corresponded to the amount of insulin added to the basal media that was used. Therefore the new islets formed from the ducts do not just store insulin but can also secrete it.

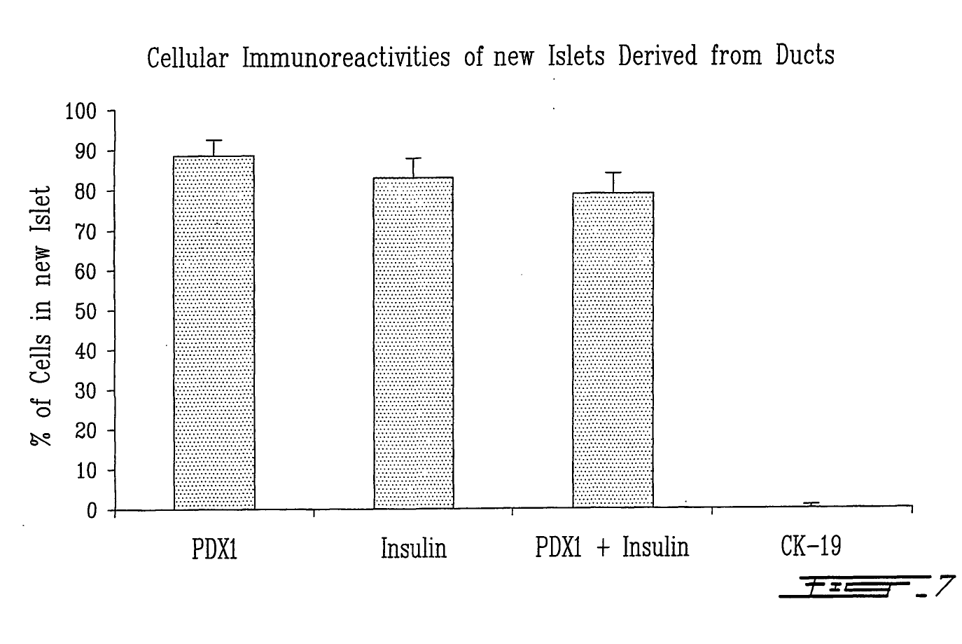

The results appearing in FIG. 7 show that about 90% of all islet cells express PDX-1, 85% express insulin, 80% express both and none of these islet cells continue to express the duct epithelial cell marker CK-19. Thus, a complete differentiation from duct to islet is obtained with associated morphological, histological and biochemical changes.

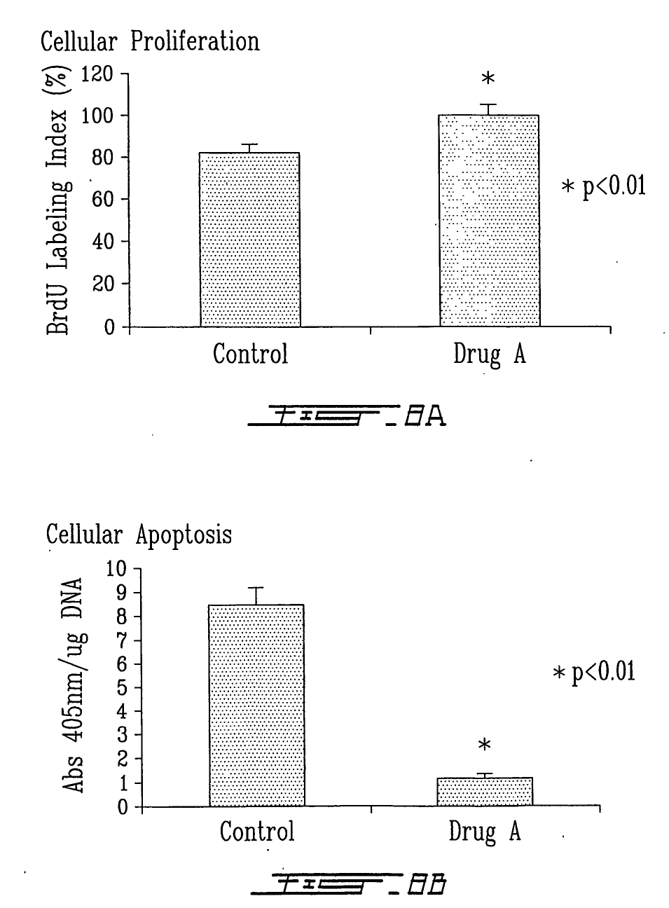

Ilotropin was noted to cause a burst in duct epithelial cell proliferation, a process known to precede new islet formation from the duct. It is shown in FIGS. 8A and 8B that the biologically active component of ilotropin, Drug A, does. indeed cause a significant increase in duct epithelial cell proliferation, as measured by BrdU labeling. In fact, almost all of the duct epithelial cells in the DRUG A group were found to be proliferative.

Clearly new cells were being formed and new islet cells were developing from the duct epithelium but were these cells surviving? There was a 90% decrease in cellular apoptosis in the DRUG A group, compared to the control, as determined by programmed cell death-specific ELISA.

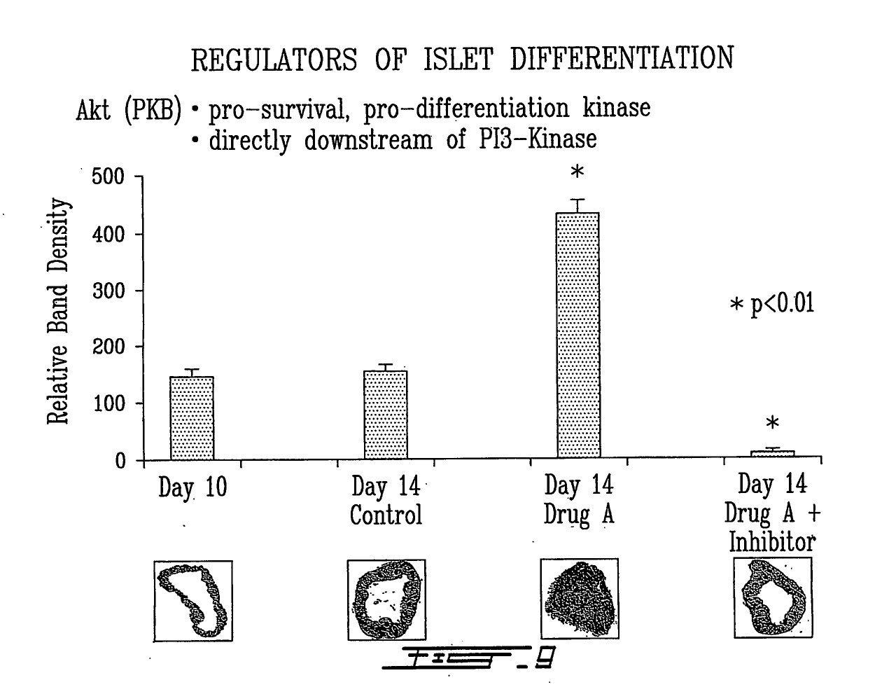

While Drug A leads to islet neogenesis from the duct, it was studied which signaling pathways mediate the drug's effects. Based on recent transgenic studies that highlight the importance of the prosurvival and prodifferentiation kinase Akt on pancreatic islet development, Akt activity relative to expression—the expression blot is shown here—was measured using Western Blot Analysis and it was found that DRUG A treatment caused a 4-fold increase in Akt activity as illustrated in FIG. 9. Interestingly, when the kinase was inhibited immediately upstream of Akt-PI3-Kinase-using Wortmannin, islet formation from the ducts was completely abrogated. Cellular proliferation and survival did not increase, PDX-1 expression did not increase, CK-19 expression persisted and islet cell hormones were not be expressed in the DRUG A/Wortmannin Group. So clearly, the PI3-Kinase signaling pathway is a major mediator of duct-to-islet differentiation.

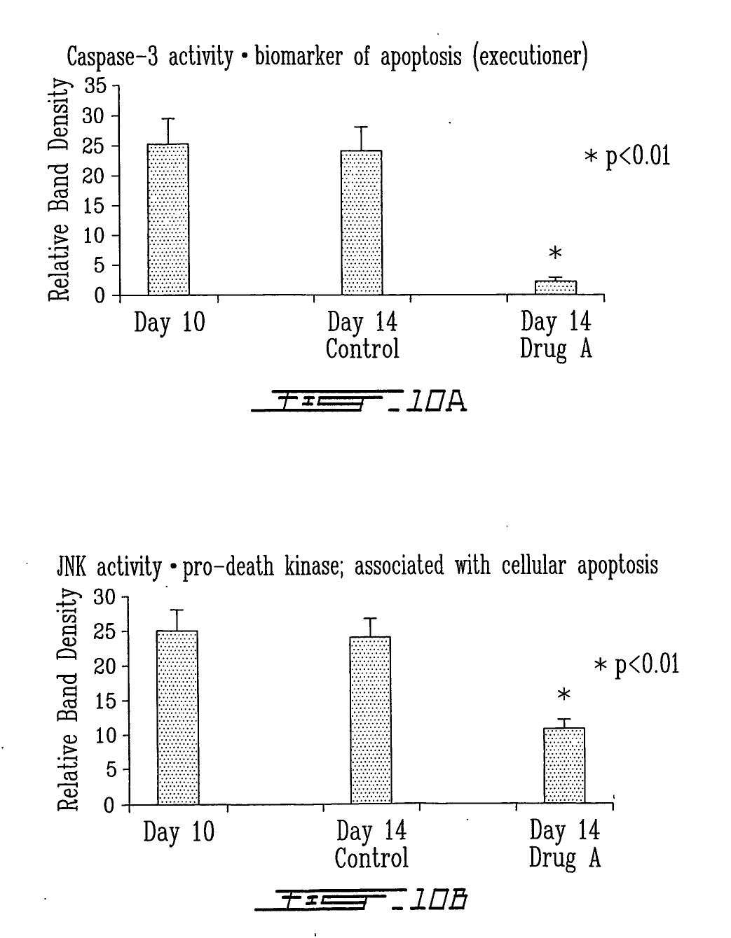

The activity of Caspase-3 was also investigated, which is an executioner of apoptosis and in fact, a biomarker of apoptosis. Its activity declined by 80% in the DRUG A group compared to the control again demonstrating DRUG A's antiapoptotic effects as illustrated in FIG. 10B.

In addition, another molecule widely associated with cell death, JNK, decreased in activity by 40% in the DRUG A group compared with the control, providing more information about the mechanism of how DRUG A mediates islet neogenesis from the duct.

In summary, Drug A is sufficient to induce islet cell neogenesis from duct epithelial cells in the adult pancreas.

This process is associated with a 3-fold increase in expression of the transcription factor PDX-1 and with a 4-fold increase in activity of the pro-survival/pro-differentiation kinase Akt. Furthermore, Drug A decreases cellular apoptosis by 80% and decreases Caspase-3 activity by over 90%.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. An in vitro method for screening agents inducing islet cell neogenesis or duct-to-islet cell transdifferentiation, which comprises the steps of:
a) expanding in vitro cells of a duct-like structure obtained by inducing cystic formation in cells in or associated with post-natal islets of Langerhans;
b) treating said expanded cells of said duct-like structure with an agent being screened; and
c) determining potency of said agent of inducing islet cell differentiation of said duct-like structure in becoming insulin-producing cells.

2. The method of claim 1, wherein step a) and step b) are concurrently effected using a solid matrix, basal feeding medium and appropriate growth factors to permit the development, maintenance and expansion of a dedifferentiated cell population with at least bipotentiality.

3. The method of claim 2, wherein said solid matrix is 3-D collagen type-1 gel matrix, said basal liquid medium is DMEM/F12 medium supplemented with EGF and cholera toxin.

4. The method of claim 1, wherein said cells are human cells.

5. A kit for carrying out the method of claim 1, which comprises:
a) a solid matrix for 3-D culture of cells;
b) a culture medium supplemented.

6. The kit of claim 8, wherein sais solid matrix is 3-D collagen type-1 gel matrix and said medium is DMEM/F12 medium supplemented with EGF and cholera toxin.

7. The kit of claim 8, which further comprises duct-like structure cells or islet cells to be transformed into duct-like structure cells.

8. An islet cell culture, which comprises insulin-producing islet cells in a suitable culture medium, wherein said islet cells are characterized.

9. The islet cell culture of claim 8, wherein said characterization is genetic, immunologic or genomic.

10. The islet cell culture of claim 9, wherein said characterization is effected using a DNA microarray analysis.

11. An in vitro method for evaluating biological effects of agents on islet cells, which comprises the steps of:
a) treating the islet cell culture of any one of claims 8 to 10 with an agent being evaluated for a time sufficient for a biological effect to be occurring; and
b) determining biological effect of said agent on islet cells by monitoring changes in insulin production compared to, a standard curve obtained with a control islet cell culture.

12. The method of claim 11, wherein said agent is selected from the group consisting of immunosuppressive agents, growth factors and anti-apoptotic agents.
